(19) United States
(12) Patent Application Publication
(10) Pub. No.: US 2008/0213797 A1
(43) Pub. Date: Sep. 4, 2008

(54) IMMUNOASSAYS EXHIBITING A REDUCTION IN PROZONE PHENOMENA

(75) Inventors: John G. Konrath, Lake Villa, IL (US); Sheng C. Lou, Libertyville, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/040,492

(22) Filed: Feb. 29, 2008

Related U.S. Application Data
(60) Provisional application No. 60/892,295, filed on Mar. 1, 2007.

(51) Int. Cl. G01N 33/53

(52) U.S. Cl. 435/7.1; 435/7.9; 435/7.92

(57) ABSTRACT

The present invention relates to immunoassays for detecting or quantifying at least one analyte of interest in a test sample. Specifically, the immunoassays of the present invention exhibit a reduction in prozone phenomena.

### RELATED APPLICATION INFORMATION

This application claims priority to U.S. Application No. 60/892,295 filed on Mar. 1, 2007, the contents of which are herein incorporated by reference.

### FIELD OF THE INVENTION

The present invention generally relates to immunoassays for detecting or quantifying at least one analyte of interest in a test sample. Specifically, the immunoassays of the present invention exhibit a reduction in an immunoassay quantitation interference known as “prozone phenomena” or “hook effect”.

### BACKGROUND OF THE PRESENT INVENTION

Immunoassays have proven to be particularly useful in testing for analytes of interest contained in test samples. In an immunoassay, the interaction of an analyte, such as an antigen, with a specific binding partner, such as an antibody, results in the formation of an analyte-binding partner complex. This complex can be detected by various measurements, such as, but not limited to, radioactivity, fluorescence, light absorption and light scattering. The results are then correlated with the presence or, absence, and ideally, with the concentration of the analyte in a test sample.

However, one problem often encountered in immunoassays is referred to as “prozone phenomena” or “hook effect”. “Prozone phenomena” refers to the characteristic shape of the analytic dilution curve and is characterized by the production of artificially low results from test samples that contain very high concentrations of the analyte of interest (such as an antigen, antibody, etc.) (See, Colin Selby, Ann. Clin. Biochem., 36:704-721 (1999)). The impact of the prozone effect on an immunoassay is exhibited by results which are but a small fraction of the true analyte concentration in the test sample.

Prozone phenomena can be caused by a number of factors. For example, in a single-step “sandwich-type” immunoassay, a capture antibody (which is an antibody that is typically immobilized on to a solid phase) is mixed with a test sample suspected of containing an analyte of interest. To this mixture an antibody containing a detectable label (hereinafter referred to as a “conjugate”) is added. In this assay, the capture antibody binds to the analyte in the test sample to form a capture antibody-analyte complex. The conjugate then binds to the capture antibody-analyte complex (the “sandwich”) and the conjugate label is detected as a measure of the analyte of interest using routine techniques known in the art. In the presence of a large excess of free analyte, all of the conjugate binds directly to the free analyte, resulting in less conjugate being available to bind to the capture antibody-analyte complex. Consequently, because less free conjugate is available to bind to the capture antibody-analyte complex, the amount of label bound to the capture antibody-analyte complex is reduced, thus reducing the amount of analyte detected. FIG. 1 shows an example of the curve generated as a result of prozone phenomena. As evidenced by FIG. 1, paradoxically, at the high end range of analyte concentration, the higher the actual analyte concentration is the lower its' measured concentration will appear.

Various techniques for reducing prozone phenomena have been proposed. For example, U.S. Pat. No. 6,183,972 describes the use of a porous material with distinct capture regions in which antibodies are immobilized. Similarly, U.S. Patent Application No. 2006/0246601 discloses a lateral flow assay device with a porous membrane and multiple zones, one of which serves as an indicator of whether the analyte in the test sample is within the hook effect region.

However, there still remains a need in the art for an immunoassay which enables an improved quantitative determination of analyte concentration in an accurate, yet simple manner.

### SUMMARY OF THE INVENTION

The present invention relates to an immunoassay for assessing at least one analyte of interest in a test sample. The immunoassay comprises the steps of:

a) incubating a first mixture for a first incubation period, the mixture comprising (1) a test sample being assessed for at least one analyte of interest; (2) a first specific binding partner that binds to the at least one analyte of interest; and (3) a second specific binding partner, wherein said second specific binding partner is labeled with a first detectable label, wherein said analyte, first specific binding partner and second specific binding partner form a first specific binding partner-analyte-second specific binding partner complex;

b) removing unbound analyte from the first mixture;

c) adding a third specific binding partner to said first mixture to form a second mixture, wherein said third specific binding partner is labeled with a second detectable label and is added to said first mixture in an amount sufficient to reduce any prozone phenomena in said immunoassay as compared to an immunoassay in which said third specific binding partner is not added;

d) incubating said second mixture for a second incubation period; and

e) detecting the first specific binding partner-analyte-second specific binding partner complex.

In the immunoassay of the present invention, the third specific binding partner is present in the second mixture in an amount which ranges from about 1% to about 50% of the amount of the second specific binding partner present in the first mixture.

Any unbound analyte can be removed from the first mixture, such as, for example, by washing the mixture after the first incubation period.

The above-described immunoassay can further comprise the optional step of washing the second mixture after the addition of the third specific binding partner.

The above-described immunoassay can further comprise the optional step of washing the second mixture after the second incubation period.

An example of a first specific binding partner that can be used in the above-described immunoassay is an antigen or an antibody. An example of an antibody that can be used is selected from the group consisting of a polyclonal antibody, a monoclonal antibody, a chimeric antibody, a human antibody, and an affinity maturated antibody.

The first specific binding partner, the second specific binding partner or the first and second specific binding partner used in the above-described immunoassay can be immobilized on a solid phase. The solid phase can be selected from the group consisting of a magnetic particle, bead, test tube, microtiter plate, cuvette, membrane, a scaffolding molecule (such as bovine serum albumin, DNA or RNA) film, filter paper, disc, and chip.

The first detectable label used in the above-described immunoassay can be selected from the group consisting of a radioactive label, an enzymatic label, a chemiluminescent label, a fluorescent label, a thermometric label, and an immuno-polymerase chain reaction label. The second detectable label used in the above-described immunoassay can be selected from the group consisting of a radioactive label, an enzymatic label, a chemiluminescent label, a fluorescent label, a thermometric label, and an immuno-polymerase chain reaction label. The first and second detectable labels can be the same labels or can be different labels.

The second specific binding partner and the third specific binding partner in the above-described immunoassay can be the same (namely, be identical) or can be different. Additionally, the second specific binding partner, the third specific binding partner or the second specific binding partner and the third specific binding partner can comprise multiple binding partners.

The first incubation period in the above-described immunoassay can comprise a period of from about 5 minutes to about 60 minutes, preferably from about 15 minutes to 30 minutes.

The second incubation period in the above-described immunoassay can comprise a period of from about 30 seconds to about 30 minutes, preferably, from about 1 minute to about 10 minutes.

In the above-described immunoassay, the immunoassay relates the amount of said first specific binding partner-analyte-second specific binding partner complex formed to the amount of the analyte in the test sample either by use of a standard curve for the analyte, or by comparison to a reference standard.

The above-described immunoassay can be used, adapted for use or performed in an automated system or in a semi-automated system.

### BRIEF DESCRIPTION OF THE FIGURES

FIG. 1 shows a representative curve generated as a result of prozone phenomena. Abscissa: Analyte Concentration (e.g., units such as ng/mL). Ordinate: Signal Amplitude (e.g., units such as Relative Light Unit counts).

FIG. 2 is a graph that shows prozone phenomena attenuation when quantifying Hepatitis B Surface Antigen (HBsAg) using a one-step immunoassay as described in more detail in Example 1. Abscissa: the concentration of HBsAg (“HBsAg ad”), ng/mL. Ordinate: Relative Light Unit (“Counts”). Symbols: solid triangles, Combination A; solid diamonds, Combination B; solid squares, Combination C.

FIG. 3 is a graph that shows prozone phenomena attenuation when quantifying HBsAg using a one-step immunoassay as described in more detail in Example 2. Abscissa: the concentration of HBsAg (“HBsAg ad”), ng/mL. Ordinate: Relative Light Unit (“Counts”). Symbols: solid squares, Combination A; solid triangles, Combination B; solid diamonds, Combination C.

### DETAILED DESCRIPTION OF THE INVENTION

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth. Unless defined otherwise herein, all technical and scientific terms used herein have the same meaning as commonly understood by one of ordinary skill in the art to which this invention belongs.

### Definitions

As used herein, the term “analyte” or “analyte of interest” as used interchangeably herein, generally refers to a substance to be detected. Analytes may include antigenic substances, haptens, antibodies, and combinations thereof. Analytes include, but are not limited to, toxins, organic compounds, DNA, RNA, proteins, peptides, microorganisms, amino acids, nucleic acids, hormones, steroids, vitamins, drugs (including those administered for therapeutic purposes as well as those administered for illicit purposes), drug intermediaries or byproducts, bacteria, virus particles and metabolites of or antibodies to any of the above substances. Specific examples of some analytes include, but are not limited to, brain natriuretic peptide (BNP) 1-32; NT-proBNP; proBNP; preproBNP; troponin I; troponin T; troponin C; antibodies or autoantibodies to cardiovascular antigens, including autoantibodies to any form of troponin; human neutrophil gelatinase-associated lipocalin (hNGAL); tacrolimus; cyclosporine; ferritin; creatinine kinase MB (CK-MB); digoxin; phenyloin; phenobarbitol; carbamazepine; vancomycin; gentamycin; theophylline; valproic acid; quinidine; luteinizing hormone (LH); follicle stimulating hormone (FSH); estradiol, progesterone; C-reactive protein; lipocalins; IgE antibodies; cytokines; vitamin B2 micro-globulin; glycated hemoglobin (Gly. Hb); cortisol; digitoxin; N-acetylprocainamide (NAPA); procainamide; antibodies to rubella, such as rubella-IgG and rubella IgM; antibodies to toxoplasmosis, such as toxoplasmosis IgG (Toxo-IgG) and toxoplasmosis IgM (Toxo-IgM); testosterone; salicylates; acetaminophen; hepatitis B virus surface antigen (HBsAg); antibodies to hepatitis B core antigen, such as anti-hepatitis B core antigen IgG and IgM (Anti-HBC); human immune deficiency virus (HIV); human T-cell leukemia virus (HTLV); hepatitis B e antigen (HbeAg); antibodies to hepatitis B e antigen (Anti-Hbe); influenza virus; thyroid stimulating hormone (TSH); thyroxine (T4); total triiodothyronine (Total T3); free triiodothyronine (Free T3); carcinoembryonic antigen (CEA); lipoproteins, cholesterol, and triglycerides; and alpha fetoprotein (AFP). Drugs of abuse and controlled substances include, but are not intended to be limited to, amphetamine; methamphetamine; barbiturates, such as amobarbital, secobarbital, pentobarbital, phenobarbital, and barbital; benzodiazepines, such as propoxy and valium; cannabinoids, such as hashish and marijuana; cocaine; fentanyl; LSD; methaqualone; opiates, such as heroin, morphine, codeine, hydromorphone, hydrocodone, methadone, oxycodone, oxymorphone and opium; phencyclidine; and propoxyphene.

As used herein, the term “antibody” refers to an immunoglobulin molecule or immunologically active portion thereof, namely, an antigen-binding portion. Examples of immunologically active portions of immunoglobulin molecules include F(ab) and F(ab′)₂ fragments which can be generated by treating an antibody with an enzyme, such as pepsin. Examples of antibodies that can be used in the present invention include, but are not limited to, polyclonal antibodies, monoclonal antibodies, chimeric antibodies, human antibodies, humanized antibodies, recombinant antibodies, single-chain Fvs (“scFv”), an affinity maturated antibody, single chain antibodies, single domain antibodies, F(ab) fragments, F(ab′) fragments, disulfide-linked Fvs (“sdFv”), and antiidiotypic (“anti-Id”) antibodies and functionally active epitope-binding fragments of any of the above.

As used herein, the terms “signal antibody”, “conjugate antibody and “conjugate” are used interchangeably herein. A signal antibody, conjugate antibody and conjugate all refer to an antibody containing a detectable label.

In terms of the detectable label, any detectable label known in the art can be used. For example, the detectable label can be a radioactive label (such as, e.g., ³H, ¹²⁵I, ³⁵S, ¹⁴C, ³²P, and ³³P), an enzymatic label (such as, e.g., horseradish peroxidase, alkaline peroxidase, glucose 6-phosphate dehydrogenase, and the like), a chemiluminescent label (such as, e.g., acridinium esters, luminal, isoluminol, thioesters, sulfonamides, phenanthridinium esters, and the like), a fluorescence label (such as, e.g., fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2^{nd} ed., Springer Verlag, N.Y. (1997) and in Haugland, Handbook of Fluorescent Probes and Research Chemicals (1996), which is a combined handbook and catalogue published by Molecular Probes, Inc., Eugene, Oreg.

As used herein, the phrase “specific binding partner,” as used herein, is a member of a specific binding pair. That is, two different molecules where one of the molecules, through chemical or physical means, specifically binds to the second molecule. Therefore, in addition to antigen and antibody specific binding pairs of common immunoassays, other specific binding pairs can include biotin and avidin, carbohydrates and lectins, complementary nucleotide sequences, effector and receptor molecules, cofactors and enzymes, enzyme inhibitors, and enzymes and the like. Furthermore, specific binding pairs can include members that are analogs of the original specific binding members, for example, an analyte-analog. Immunoreactive specific binding members include antigens, antigen fragments, antibodies and antibody fragments, both monoclonal and polyclonal and complexes thereof, including those formed by recombinant DNA molecules.

As used herein, the term “test sample” generally refers to a biological material suspected of containing and/or being tested for an analyte of interest. The test sample may be derived from any biological source, such as, a physiological fluid, including, but not limited to, whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid, semen and so forth. Besides physiological fluids, other liquid samples may be used such as water, food products, and so forth, for the performance of environmental or food production assays. In addition, a solid material suspected of containing the analyte may be used as the test sample. The test sample may be used directly as obtained from the biological source or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release the analyte.

### Prozone Phenomenon-Remedied Immunoassay

In general, the present invention relates to an immunoassay for assessing (e.g., detecting or quantifying) at least one analyte of interest in a test sample, where an immunoassay quantitation interference known as “prozone phenomena” or “hook effect” is remedied (e.g., suppressed, reduced in amount, or altogether eliminated).

In a typical “one-step” immunoassay known in the art, an analyte of interest, a first specific binding partner, such as a capture antibody, and a second specific binding partner that has been labeled with a detectable label, such as a conjugate antibody, are all simultaneously added to form a reaction mixture. The resulting mixture is then allowed to incubate and any unbound analyte removed from said mixture. Optionally, a diluent can be added after the removal of any unbound analyte. The concentration of the analyte is then determined using routine techniques known to those skilled in the art, including, but not limited to, the use of a standard curve for the analyte and/or comparison to a reference standard. Prozone phenomena may occur when an excess of free (i.e., unbound) analyte is present in the test sample. Specifically, excess unbound analyte may saturate free conjugate antibody present in the reaction mixture to such an extent that not enough unsaturated free conjugate antibody remains in the mixture to bind to and label the capture antibody-analyte complexes.

Generally, as will be described in more detail herein, according to the present invention, prozone phenomena can be reduced or eliminated by the addition of a third specific binding partner to the reaction mixture. This third specific binding partner is labeled with a detectable label and is added to the reaction mixture after any unbound analyte has been removed from the reaction mixture. Specifically, the third specific binding partner binds the analyte of interest and not to either the first specific binding partner or the second specific binding partner. For example, if the analyte of interest is a protein or antigen, the third specific binding partner can be an antibody that binds to said protein or antigen. Alternatively, if the analyte of interest is an antibody, the third specific binding partner can be a protein or antigen that binds to said antibody. After the addition of the labeled third specific binding partner, prozone phenomena is remedied (e.g., suppressed, reduced in amount, or altogether eliminated) because the addition of the labeled third specific binding partner serves to replace any of the labeled second specific binding partner which might have been depleted by the excess of free analyte in the test sample.

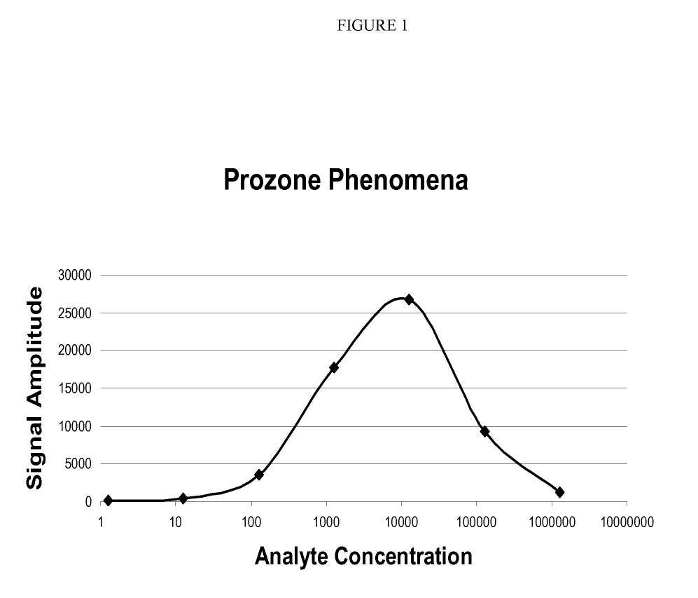

Such remedy of prozone phenomena as described herein includes any dampening, attenuation, or reduction in prozone phenomena as compared to a comparable assay in which a third specific binding partner is not included. Dampening or attenuation refers generally to a “smoothening” of the analyte concentration versus signal amplitude curve, such that the decrease in signal amplitude that otherwise would be obtained with high analyte concentration (i.e., absent the addition of the third specific binding partner, e.g., as exhibited in FIG. 1) does not occur at all, or is reduced or eliminated for some or all high analyte concentrations. Such reduction in amount can be any reduction observed for only single analyte concentrations, or over a range of analyte concentrations. Such reduction can range from about 2% to about 100% (e.g., with 100% reduction evidencing “elimination” of the phenomenon for that analyte concentration), particularly reduction greater than about 5% (e.g., from about 5% to about 98%, or from about 5% to about 95%, or from about 5% to about 90%), especially reduction greater than about 10% (e.g., from about 10% to about 90%, or from about 10% to about 85%, or from about 10% to about 80%), and optionally reduction greater than about 30% (e.g., e.g., from about 30% to about 80%, or from about 30% to about 70%, or from about 30% to about 60%).

In one embodiment, the present invention relates to a one-step immunoassay. The immunoassay comprises a first reaction mixture comprising at least one analyte of interest, a first specific binding partner that binds to the at least one analyte and a second specific binding partner wherein the second specific binding partner is labeled with a first detectable label. The at least one analyte of interest, a first specific binding partner and a second specific binding partner can each be added in any order, sequentially or simultaneously. The immunoassay further comprises adding a third specific binding partner to the first reaction mixture, thus forming a second reaction mixture, wherein the third specific binding partner is labeled with a second detectable label. The first detectable label and the second detectable label can be the same or different. The third specific binding partner can be added to the immunoassay following incubation of the first reaction mixture containing the first specific binding partner, the at least one analyte and the second specific binding partner. Prior to the addition of the third specific binding partner, any unbound analyte contained in the first reaction mixture is removed, using routine techniques known in the art, such as washing. Preferably, the first specific binding partner is a capture antibody and the second specific binding partner is the conjugate antibody.

Accordingly, in another embodiment, the present invention relates to an immunoassay comprising the steps of:

a) incubating, for a first incubation period, a first mixture comprising (1) a test sample being assessed for at least one analyte of interest; (2) a first specific binding partner that binds to the at least one analyte of interest; and (3) a second specific binding partner, wherein said second specific binding partner is labeled with a first detectable label, wherein said analyte, first specific binding partner and second specific binding partner form a first specific binding partner-analyte-second specific binding partner complex;

b) removing unbound analyte from the first mixture;

c) adding a third specific binding partner to said first mixture to form a second mixture, wherein said third specific binding partner is labeled with a second detectable label and is added to said first mixture in an amount sufficient to reduce any prozone phenomena in said immunoassay as compared to an immunoassay in which said third specific binding partner is not added;

d) incubating said second mixture for a second incubation period; and

e) determining the amount of said first specific binding partner-analyte-second specific binding partner complex.

The test sample containing the at least one analyte of interest, the first specific binding partner and the second specific binding partner can be added in any order, sequentially or simultaneously.

The amount of the third specific binding partner sufficient to reduce prozone phenomena in the immunoassays described herein can vary depending on a variety of factors, including, but not limited to, whether the assay is intended to be quantitative or qualitative for a particular analyte of interest. In general, for a qualitative assay, the amount of the third specific binding partner used should be high enough so that a false negative result is not obtained. A false negative result occurs when the analyte of interest is identified as being absent in a test sample when, in fact, the analyte is actually present in the test sample. In general, for a quantitative assay, the amount added should be sufficient to prevent the signal from being less than the highest calibrator ensuring that a dilution of the sample is needed to determine analyte amount and to avoid underquantifying the amount of the analyte. Preferably, the amount of third specific binding partner used in the immunoassays described herein is from about 1% to about 50% of the amount of the second specific binding partner.

In the immunoassays of the present invention, the amount of said first specific binding partner-analyte-second specific binding partner complex formed is related to the amount of the analyte in the test sample either by use of a standard curve for the analyte, or by comparison to a reference standard. The standard curve can be generated using serial dilutions of analyte of interest of known concentration, by mass spectroscopy, gravimetrically and by other techniques known in the art. Optionally the amount of the analyte in the test sample is quantitated by measuring the amount of the second detectable label.

After the first incubation period, the unbound analyte can be removed using routine techniques known in the art, such as washing.

Additionally, in the immunoassays of the present invention the first detectable label and the second detectable label can be the same (identical) or different.

Additionally, in another embodiment, the immunoassay may further comprise an additional step of washing the second mixture after the addition of the third specific binding partner.

Alternatively, in yet another embodiment, the immunoassay may comprise the step of washing the second mixture after the second incubation period.

In the immunoassays described herein, optionally the first specific binding partner, the second specific binding partner, the third specific binding partner, the first specific binding partner and the second specific binding partner, the first specific binding partner and the third specific binding partner, the second specific binding partner and the third specific binding partner or the first specific binding partner, the second specific binding partner and the third specific binding partner are immobilized on a solid phase. The solid phase can be any material known to those of ordinary skill in the art to which the specific binding partners, such as, but not limited to, antibodies or antigens, can be attached. Examples of solid phases that can be used, include, but are not limited to, a test well in a microtiter plate, nitrocellulose, nylon, a bead or a disc (which can be made out of glass, fiberglass, latex, plastic or a paper material), a gel (for example, a gel through which the polypeptides have been run and which is subsequently dried), a scaffolding molecule (such as, but not limited to, bovine serum albumin, DNA or RNA) or a strip, disc or sheet (which can be made out of nitrocellulose, nylon, plastic or paper). Optionally, the first specific binding partner, the second specific binding partner, the third specific binding partner, the first specific binding partner and the second specific binding partner, the first specific binding partner and the third specific binding partner, the second specific binding partner and the third specific binding partner or the first specific binding partner, the second specific binding partner and the third specific binding partner can be bound to the solid phase by adsorption, by covalent bonding using a chemical coupling agent or by other means known in the art, provided that such binding does not interfere with the ability of any of the specific binding partners (namely, the first specific binding partner, the second specific binding partner, the third specific binding partner, the first specific binding partner and the second specific binding partner, the first specific binding partner and the third specific binding partner, the second specific binding partner and the third specific binding partner or the first specific binding partner, the second specific binding partner and the third specific binding partner) to bind to the analyte of interest. Moreover, if necessary, the solid phase can be derivatized to allow reactivity with various functional groups on any of the specific binding partners (namely, the first specific binding partner, the second specific binding partner, the third specific binding partner, the first specific binding partner and the second specific binding partner, the first specific binding partner and the third specific binding partner, the second specific binding partner and the third specific binding partner or the first specific binding partner, the second specific binding partner and the third specific binding partner). Such derivatization requires the use of certain coupling agents such as, but not limited to, maleic anhydride, N-hydroxysuccinimide and 1-ethyl-3-(3-dimethylaminopropyl)carbodiimide.

In the immunoassays described herein, the second specific binding partner and the third specific binding partner can be the same or they can be different. For example, the second specific binding partner and the third specific binding partner can each be the same antibody or antigen or each can be a different antibody and antigen. The important characteristic of the third specific binding partner is that its addition to an immunoassay results in reduction of prozone phenomena.

Moreover, the second specific binding partner, third specific binding partner or the second specific binding partner and third specific binding partner can comprise multiple binding partners. For example, the binding partners may comprise a mixture of antibodies at the same or different concentrations.

The length of the first and second incubation periods described in the immunoassays herein can vary depending on a variety of factors, including, but not limited to, the identity of specific binding partners. In general, a person of ordinary skill in the art will be able to readily determine the needed length of time, as the incubations are similar as in known immunoassays. In a preferred embodiment, the first incubation period is from about 5 minutes to about 60 minutes. In a more preferred embodiment, the first incubation period is from about 15 minutes to about 30 minutes. In a preferred embodiment, the second incubation period is from about 30 seconds to about 30 minutes. In a more preferred embodiment, the second incubation period is from about 1 minute to about 10 minutes. Generally, the length of the second incubation period may be shorter than the length of the first incubation period, as the solid phase antibody will be saturated with analyte, and therefore, less time is required for the second incubation conjugate antibody to form a complex with the analyte/solid phase antibody complex.

In a preferred embodiment, the concentration of the third specific binding partner is lower compared to the concentration of the second specific binding partner. Because the first specific binding partner is saturated with analyte, the third specific binding partner is able to achieve a high degree of binding and labeling of the first specific binding partner-analyte-second specific binding partner complex. In addition, the use of low concentration third specific binding partner results in lower background signal (hence a lower signal to noise ratio), allowing higher sensitivity analyte detection.

Thus, among other things, provided herein is an improvement of an immunoassay of test sample for analyte wherein an analyte of interest present in said test sample is captured by a capture antibody and detected by a first antibody conjugate by forming a complex of capture antibody, analyte and first antibody conjugate, the improvement comprising a further step with the addition of second antibody conjugate that binds to said analyte of interest. The further labeled conjugate can be can be added as an additional incubation following a conventional one-step immunoassay. Alternately, the second incubation can be the existing second incubation of a modified two-step immunoassay. Optimally this addition is done following removal of any unbound analyte from the mixture. Of course, this improved assay can be done wherein antigen instead of antibody is employed to complex analyte using modifications that are well known to those skilled in the art.

The invention as described herein also can be adapted for use in a variety of automated and semi-automated systems (including those wherein the solid phase comprises a microparticle), as described, e.g., in U.S. Pat. Nos. 5,089,424 and 5,006,309, and as, e.g., commercially marketed by Abbott Laboratories (Abbott Park, Ill.) including but not limited to Abbott's ARCHITECT®, AxSYM, IMX, PRISM, and Quantum II instruments, as well as other platforms. Moreover, the invention optionally is adaptable for the Abbott Laboratories commercial Point of Care (i-STAT™) electrochemical immunoassay system for performing sandwich immunoassays. Immunosensors, and their methods of manufacture and operation in single-use test devices are described, for example in, U.S. Pat. No. 5,063,081, US Patent Application 20030170881, US Patent Application 20040018577, US Patent Application 20050054078, and US Patent Application 20060160164, which are incorporated in their entireties by reference for their teachings regarding same.

The invention will further be illustrated through specific examples. These examples represent only preferred embodiments of the invention and are not meant to be limiting.

### EXAMPLE 1

### Prozone Phenomena Attenuation when Quantifying HBsAg Using a One Step Immunoassay

An automated ARCHITECT® system (Abbott Laboratories, Abbott Park, Ill.) was used to perform an immunoassay that would quantitate Hepatitis B Surface Antigen (HBsAg) in a unit of human plasma that contained a 1.27 mg/mL of HBsAg, which is considered to be a high concentration of HBsAg in a test sample.

Dilutions of a unit of human plasma containing HBsAg (1.27 mg/mL-127 pg/mL were prepared using 10-fold dilution steps. Testing was performed in reaction vessels that are used for individual tests in the automated ARCHITECT® system. All the described steps were performed in the ARCHITECT® instrument. Each HBsAg dilution was dispensed in the amount of 75 μL into individual reaction vessels. At the same time, 0.075% 1-ethyl-3-[3-dimethylaminopropyl]carbodiimide hydrochloride (“EDAC”) coated magnetic microparticles (Polymer Science, Monticello, Ind.) (50 μL) coated with anti-HBsAg monoclonal antibodies (Abbott Laboratories, Abbott Park, Ill.), and anti-HBsAg antibodies labeled with acridinium, either 100 ng/mL or 0.0 ng/mL (Abbott Laboratories, Abbott Park, Ill.) were dispensed in the amount of 50 μL each to the same reaction vessel. The reaction vessel was then vortexed to mix the sample and reactants. Each reaction vessel containing the reaction mixture was incubated for 18 minutes at 37° C.

During this incubation, the HBsAg in the sample was captured by the anti-HBsAg monoclonal antibodies coated onto the magnetic microparticles.

The anti-HBsAg acridinium labeled antibodies also bound to HBsAg that was captured by the magnetic microparticles. This formed a microparticle-HBsAg-labeled antibody, complex.

Upon completion of the 18 minute incubation, the microparticle-HBsAg-labeled antibody complexes were magnetically-captured, and immobilized, onto the side of the reaction vessel. The immobilized microparticle-HBsAg-labeled antibody complexes were then washed by alternately aspirating the liquid from the vessel, and then adding wash buffer (the wash buffer contains PBS, Brij-35 (Polyoxyethyleneglycol dodecyl ether) and has a pH of about 6.8) into the reaction vessel (1 mL wash buffer, repeated 4 times).

This process removed unbound sample, and unbound assay reactants, from the reaction mixture. The magnetically-captured microparticle-HBsAg-labeled antibody complexes formed during the 18 minute incubation remained in the reaction vessel.

During the second incubation (4 minutes), the captured magnetic microparticle-HBsAg-labeled antibody complexes were released from the magnet. Acridinium labeled anti-HBsAg antibody (same as used above) (45 or 0.0 ng/mL (the 0.0 ng/mL means that only diluent is used—no third specific binding partner is used)) was then dispensed in the amount of 50 μL to the reaction vessel containing the microparticle-HBsAg-labeled antibody complexes. This reaction mixture is then vortexed to disperse the microparticles and mix the reactants. The reaction mixture was incubated for 4 minutes at 37° C.

Upon completion of the 4 minute incubation, the microparticle-HBsAg-labeled antibody complexes are magnetically captured again. They are then repeatedly washed with buffer (1 mL wash buffer, repeated 4 times) (the same wash buffer described above). This removes unbound labeled anti-HBsAg antibody.

The magnetically-captured microparticle-HBsAg complexes are then released.

The acridinium label (Abbott Laboratories, Abbott Park, Ill.) is then triggered to emit light. This is accomplished by adding a low pH (pH 1) buffer containing H₂O₂ (1.32%) (Abbott Laboratories, Abbott Park) in the amount of about 100 μL to the microparticle complexes and vortexing. The addition of this buffer releases the anti-HBsAg monoclonals labeled with acridinium (Abbott Laboratories, Abbott Park) that had bound to HBsAg captured by the microparticles.

The magnetic microparticles are then magnetically-captured leaving the released HBsAg-labeled acridinium in the reaction mixture solution. This is followed by addition of about 300 μL of a pH 13 buffer which “triggers” light production from the acridinium released into the solution.

The amount of light generated (which is measured in “Relative Light Units” (RLU)) is used to determine the quantity of HBsAg present in the sample.

The dilutions of the HBsAg samples were tested with the three combinations of anti-HBsAg antibodies labeled with acridinium (Acrd). Combination (A) used anti-HBsAg antibodies labeled with acridinium only during the first incubation. Combination (B) used anti-HBsAg antibodies labeled with acridinium during the first and second incubation. Combination (C) used anti-HBsAg antibodies labeled with acridinium only during the second incubation. Specifically, each of these three combinations are described in more detail below and in Table 1.

### Anti-HBsAg Acridinium Combinations Tested:

Combination (A)-[“100_{—}/0”] anti-HBsAg Acrd:
100 ng/mL anti-HBsAg Acrd added to incubation one.
0 ng/mL anti-HBsAg Acrd added to incubation two.

Combination (B)-[“100/45”] anti-HBsAg Acrd:
100 ng/mL anti-HBsAg Acrd added to incubation one.
45 ng/mL anti-HBsAg Acrd added to incubation two.

Combination (C)-[“0/45” anti-HBsAg Acrd:
0 ng/mL anti-HBsAg Acrd added to incubation one.
45 ng/mL anti-HBsAg Acrd added to incubation two.

TABLE 1
Counts produced by each test condition and the sample tested versus
HBsAg concentration.

| | Combination | Combination | Combination |
|---|---|---|---|
| | (A) | (B) | (C) |
| Anti-HBsAg Acrd Conjugate, | 100 ng/mL | 100 ng/mL | 0 ng/mL |
| Incubation 1 | | | |
| Anti-HBsAg Acrd Conjugate, | 0 ng/mL | 45 ng/mL | 45 ng/mL |
| Incubation 2 | | | |

| HBsAg, Acrd | | | |
|---|---|---|---|
| ng/mL | Counts | Counts | Counts |
| 1270000 | 125806 | 2465413 | 2320352 |
| 127000 | 868632 | 2854795 | 2386725 |
| 12700 | 2559818 | 3656575 | 2429112 |
| 1270 | 2197466 | 2856264 | 1630232 |
| 127 | 490786 | 656667 | 369959 |
| 12.7 | 59065 | 82985 | 45760 |
| 1.27 | 6088 | 8348 | 4888 |
| 0.127 | 715 | 1020 | 609 |

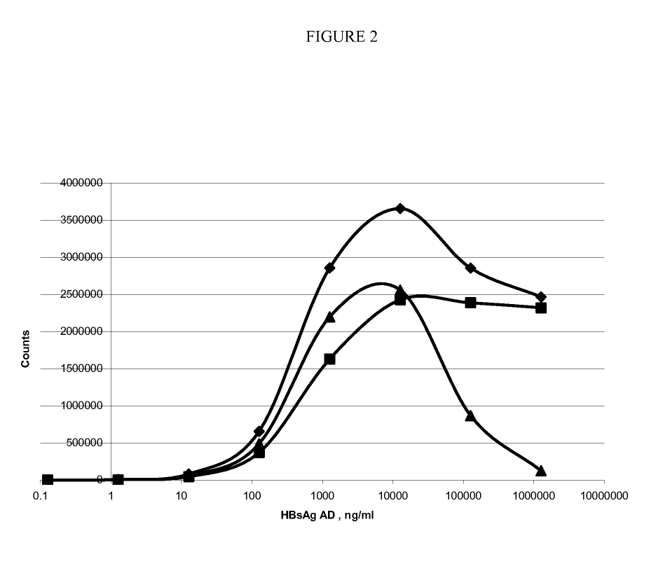

FIG. 2 is a graph of the counts produced by each test condition and the sample tested versus HBsAg concentration.

These results confirm that using acridinium labeled antibodies only in the first incubation results in an obvious prozone effect that reduces the 1.27 mg/mL HBsAg sample RLUs down to 128,506 counts. This compares to 2,559,818 counts at 12,700 ng/mL HBsAg. If the 125,806 counts for the HBsAg 1.27 mg/mL were interpreted by comparing these counts to the ascending portion of this graph, the 1.27 mg/mL HBsAg dilution would be quantitated as about ˜29.7 ng/mL. This demonstrates significant immunoassay signal suppression due to the prozone phenomena.

The addition of anti-HBsAg acridinium labeled antibodies into the second incubation, and first incubation, significantly reduces the hook effect by limiting the prozone phenomena and resulting in 2,465,413 counts for 1.27 mg/mL. This would raise the quantitation of the 1.27 mg/mL HBsAg sample up to 1067 ng/mL, a 35-fold increase in quantitation.

The addition of anti-HBsAg acridinium labeled antibodies into only the second incubation illustrates the mechanism of the prozone phenomena suppression. The addition of anti-HBsAg acridinium in the second incubation provides signal that is not subject to prozone phenomena. Even if the entire signal from the first incubation was eliminated due to the prozone phenomena, the signal would not fall beneath the level provided by anti-HBsAg acridinium labeled antibodies.

### EXAMPLE 2

### Prozone Phenomena Attenuation when Quantifying HBsAg Using a One Step Immunoassay

An automated ARCHITECT® system (Abbott Laboratories, Abbott Park, Ill.) was used to perform an immunoassay that would quantitate Hepatitis B Surface Antigen (HBsAg) in a unit of human plasma that contained a 1.27 mg/mL of HBsAg, which is considered to be a high concentration of HBsAg in a test sample. In this example, the amount of the anti-HBsAg antibodies labeled with acridinium was substantially reduced over the amount employed in Example 1. Namely rather than using either 0 ng/mL or 100 ng/mL in the first incubation and 0 ng/mL or 45 ng/mL in the second incubation, an amount of up to only 1.5 ng/mL was tested in each of the first and second incubations.

Dilutions of a unit of human plasma containing HBsAg (1.27 mg/mL-1.27 ng/mL were prepared using 10-fold dilution steps. Testing was performed in reaction vessels that are used for individual tests in the automated ARCHITECT® system. All the described steps were performed in the ARCHITECT® instrument. Each HBsAg dilution was dispensed in the amount of 75 μL into individual reaction vessels. At the same time, 0.075% EDAC coated magnetic microparticles (Polymer Science, Monticello, Ind.) (50 μL) coated with anti-HBsAg monoclonal antibodies (Abbott Laboratories, Abbott Park, Ill.), and anti-HBsAg antibodies labeled with acridinium, either 1.5 ng/mL or 0.0 ng/mL (Abbott Laboratories, Abbott Park, Ill.) were dispensed in the amount of 50 μL each to the same reaction vessel. The reaction vessel was then vortexed to mix the sample and reactants. Each reaction vessel containing the reaction mixture was incubated for 18 minutes at 37° C.

During this incubation, the HBsAg in the sample was captured by the anti-HBsAg monoclonal antibodies coated onto the magnetic microparticles.

The anti-HBsAg acridinium labeled antibodies also bound to HBsAg that was captured by the magnetic microparticles. This formed a microparticle-HBsAg-labeled antibody, complex.

Upon completion of the 18 minute incubation, the microparticle-HBsAg-labeled antibody complexes were magnetically captured, and immobilized, onto the side of the reaction vessel. The immobilized microparticle-HBsAg-labeled antibody complexes were then washed by alternately aspirating the liquid from the vessel, and then adding wash buffer (the wash buffer contains PBS, Brij-35 (Polyoxyethyleneglycol dodecyl ether) and has a pH of about 6.8) into the reaction vessel (1 mL wash buffer, repeated 4 times).

This process removed unbound sample, and unbound assay reactants, from the reaction mixture. The magnetically captured microparticle-HBsAg-labeled antibody complexes formed during the 18 minute incubation remained in the reaction vessel.

During the second incubation (4 minutes), the captured magnetic microparticle-HBsAg-labeled antibody complexes were released from the magnet. Acridinium labeled anti-HBsAg antibody (same as used above) (0.15 or 0.0 ng/mL) was then dispensed in the amount of 50 μL to the reaction vessel containing the microparticle-HBsAg-labeled antibody complexes. This reaction mixture is then vortexed to disperse the microparticles and mix the reactants. The reaction mixture was incubated for 4 minutes at 37° C.

Upon completion of the 4 minute incubation, the microparticle-HBsAg-labeled antibody complexes are magnetically captured again. They are then repeatedly washed with buffer (1 mL wash buffer, repeated 4 times) (the same wash buffer described above). This removes unbound labeled anti-HBsAg antibody.

The magnetically captured microparticle-HBsAg complexes are then released.

The acridinium label (Abbott Laboratories, Abbott Park, Ill.) is then triggered to emit light. This is accomplished as described in Example 1, namely by adding a low pH (pH 1) buffer containing H₂O₂ (1.32%) (Abbott Laboratories, Abbott Park) in the amount of about 100 μL to the microparticle complexes and vortexing.

The magnetic microparticles are then magnetically-captured leaving the released HBsAg-labeled acridinium in the reaction mixture solution. This is followed by addition of about 300 μL of a pH 13 buffer which “triggers” light production from the acridinium released into the solution.

As in Example 1, the amount of light generated is measured in RLUs and is used to determine the quantity of HBsAg present in the sample.

The dilutions of the HBsAg samples were tested with three combinations of anti-HBsAg antibodies labeled with acridinium. Combination (A) used anti-HBsAg antibodies labeled with acridinium only during the first incubation. Combination (B) used anti-HBsAg antibodies labeled with acridinium during the first and second incubation. Combination (C) used anti-HBsAg antibodies labeled with acridinium only during the second incubation. Each of these three combinations are described in more detail below and in Table 2.

### Anti-HBsAg Acridinium Combinations Tested:

Combination (A)-[“1.5_{—}/0”] anti-HBsAg Acrd:
1.5 ng/mL anti-HBsAg Acrd added to incubation one.
0 ng/mL anti-HBsAg Acrd added to incubation two.

Combination (B)-[“1.5/0.15”] anti-HBsAg Acrd:
1.5 ng/mL anti-HBsAg Acrd added to incubation one.
0.15 ng/mL anti-HBsAg Acrd added to incubation two.

Combination (C)-[“0/0.15”] anti-HBsAg Acrd:
0 ng/mL anti-HBsAg Acrd added to incubation one.
0.15 ng/mL anti-HBsAg Acrd added to incubation two.

TABLE 2
Counts produced by each test condition and the sample tested versus
HBsAg concentration.

| | Combination | Combination | Combination |
|---|---|---|---|
| | (A) | (B) | (C) |
| Anti-HBsAg Acrd, Conjugate, | 1.5 ng/mL | 1.5 ng/mL | 0 ng/mL |
| Incubation 1 | | | |
| Anti-HBsAg Acrd, Conjugate, | 0 ng/mL | 0.15 ng/mL | 0.15 ng/mL |
| Incubation 2 | | | |

| HBsAg, Acrd | | | |
|---|---|---|---|
| ng/mL | Counts | Counts | Counts |
| 1270000 | 4387 | 21072 | 16789 |
| 127000 | 32639 | 49974 | 17445 |
| 12700 | 91967 | 109466 | 17006 |
| 1270 | 59983 | 67854 | 9043 |
| 127 | 12863 | 14010 | 2000 |
| 12.7 | 1473 | 1632 | 293 |
| 1.27 | 230 | 261 | 105 |

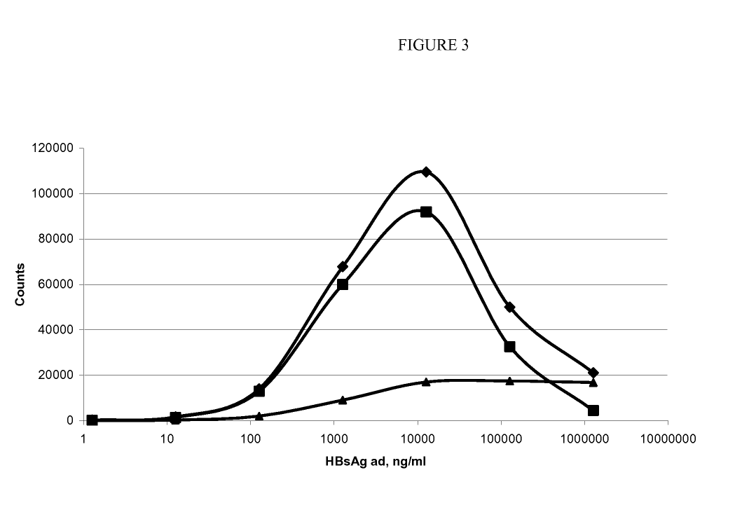

FIG. 3 is a graph of the counts produced by each test condition and the sample tested versus HBsAg concentration.

The results confirm that using acridinium labeled antibodies only in the first incubation results in an obvious prozone effect that reduces the 1.27 mg/mL HBsAg sample RLUs down to 4387 counts. This compares to 91967 counts at 12,700 ng/mL HBsAg. If the 4387 counts for the HBsAg 1.27 mg/mL were interpreted by comparing to these counts to the ascending portion of this graph the 1.27 mg/mL HBsAg dilution would be quantitated as about ˜41.9 ng/mL. This demonstrates significant immunoassay signal suppression due to the prozone phenomena.

The addition of anti-HBsAg acridinium labeled antibodies into the second incubation, and first incubation, significantly reduces the hook effect by limiting the prozone phenomena resulting in 21072 counts for 1.27 mg/mL. This would raise the quantitation of the 1.27 mg/mL HBsAg sample up to 276.9 ng/mL, a 6.6-fold increase in quantitation.

The addition of anti-HBsAg acridinium labeled antibodies into only the second incubation illustrates the mechanism of the prozone phenomena suppression. The addition of anti-HBsAg acridinium in the second incubation provides signal that is not subject to prozone phenomena. Even if the entire signal from the first incubation was eliminated due to the prozone phenomena, the signal would not fall beneath the level provided by anti-HBsAg acridinium labeled antibodies added to the second incubation.

Moreover, these results confirm that the remedy for prozone phenomena as described herein can be obtained with a reduced amount of antibody conjugate employed during a brief second incubation step without substantial loss of analyte detection sensitivity, and with at most a minimal increase in background non-specific signal.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. An immunoassay for assessing at least one analyte of interest in a test sample, the immunoassay comprising the steps of:
a) incubating a first mixture for a first incubation period, the mixture comprising (1) a test sample being assessed for at least one analyte of interest; (2) a first specific binding partner that binds to the at least one analyte of interest; and (3) a second specific binding partner labeled with a first detectable label, wherein said analyte, first specific binding partner and second specific binding partner form a first specific binding partner-analyte-second specific binding partner complex;
b) removing unbound analyte from the first mixture;
c) adding a third specific binding partner to said first mixture to form a second mixture, wherein said third specific binding partner is labeled with a second detectable label and is added to said first mixture in an amount sufficient to reduce any prozone phenomena in said immunoassay as compared to an immunoassay in which said third specific binding partner is not added;
d) incubating said second mixture for a second incubation period; and
e) detecting said first specific binding partner-analyte-second specific binding partner complex.

2. The immunoassay of claim 1, wherein said third specific binding partner is present in the second mixture in an amount which ranges from about 1% to about 50% of the amount of the second specific binding partner present in the first mixture.

3. The immunoassay of claim 1, further comprising the step of washing the second mixture after the addition of the third specific binding partner.

4. The immunoassay of claim 1, further comprising the step of washing the second mixture after the second incubation period.

5. The immunoassay of claim 1, wherein said first specific binding partner is an antibody or an antigen.

6. The immunoassay of claim 5, wherein said antibody is selected from the group consisting of a polyclonal antibody, a monoclonal antibody, a chimeric antibody, a human antibody, and an affinity maturated antibody.

7. The immunoassay of claim 1, wherein said first specific binding partner is immobilized on a solid phase.

8. The immunoassay of claim 7, wherein the solid phase is selected from the group consisting of a magnetic particle, bead, test tube, microtiter plate, cuvette, membrane, a scaffolding molecule, film, filter paper, disc, and chip.

9. The immunoassay of claim 1, wherein the first detectable label is selected from the group consisting of a radioactive label, an enzymatic label, a chemiluminescent label, a fluorescent label, a thermometric label, and an immuno-polymerase chain reaction label.

10. The immunoassay of claim 1, wherein the second detectable label is selected from the group consisting of a radioactive label, an enzymatic label, a chemiluminescent label, a fluorescent label, a thermometric label, and an immuno-polymerase chain reaction label.

11. The immunoassay of claim 1, wherein the first detectable label and the second detectable label are the same.

12. The immunoassay of claim 1, wherein the first detectable label and the second detectable label are different.

13. The immunoassay of claim 1, wherein the second specific binding partner is immobilized on a solid phase.

14. The immunoassay of claim 13, wherein the solid phase is selected from the group consisting of a magnetic particle, bead, test tube, microtiter plate, cuvette, membrane, a scaffolding molecule, film, filter paper, disc, and chip.

15. The immunoassay of claim 1, wherein the second specific binding partner and the third specific binding partner are the same.

16. The immunoassay of claim 1, wherein the second specific binding partner and the third specific binding partner are different.

17. The immunoassay of claim 1, wherein the second specific binding partner comprises multiple binding partners.

18. The immunoassay of claim 1, wherein the third specific binding partner comprises multiple binding partners.

19. The immunoassay of claim 1, wherein the first incubation period comprises a period of from about 5 minutes to about 60 minutes.

20. The immunoassay of claim 19, wherein the first incubation period comprises a period of from about 15 minutes to about 30 minutes.

21. The immunoassay of claim 1, wherein the second incubation period comprises a period of from about 30 seconds to about 30 minutes.

22. The immunoassay of claim 21, wherein the second incubation period comprises a period of from about 1 minute to about 10 minutes.

23. The immunoassay of claim 1, wherein the unbound analyte is removed from the first mixture by washing.

24. The immunoassay of claim 1, wherein the immunoassay relates an amount of said first specific binding partner-analyte-second specific binding partner complex formed to the amount of the analyte in the test sample either by use of a standard curve for the analyte, or by comparison to a reference standard.

25. The immunoassay of claim 1, wherein the amount of the analyte in the test sample is quantitated by measuring the amount of the second detectable label.

26. The immunoassay of claim 1, wherein said immunoassay is adapted for use in an automated system or semi-automated system.

27. In an improvement of an immunoassay of test sample for analyte wherein an analyte of interest present in said test sample is captured by a capture antibody and detected by a first antibody conjugate by forming a complex of capture antibody, analyte and first antibody conjugate,
the improvement comprising a further step with the addition of second antibody conjugate that binds to said analyte of interest.

28. In the improvement of claim 27, wherein said immunoassay is adapted for use in an automated system or semi-automated system.
